(19) United States
(12) Patent Application Publication
(10) Pub. No.: US 2006/0057732 A1
(43) Pub. Date: Mar. 16, 2006

(54) Methods in the diagnosis of pulmonary embolism

(75) Inventors: Per Agvald, Bagarmossen (SE); Dag Linnarsson, Stocksund (SE); Christofer Adding, Akersberga (SE); Lars Gustafsson, Hasselby (SE); Kristofer Nilsson, Huskvarna (SE)

Correspondence Address: MORRISON & FOERSTER LLP, 425 MARKET STREET, SAN FRANCISCO, CA, 94105-2482, US

(73) Assignee: Aerocrine AB, Solna (SE)

(21) Appl. No.: 11/225,313

(22) Filed: Sep. 12, 2005

(30) Foreign Application Priority Data
Sep. 14, 2004 (SE) SE-0402220-8

(51) Int. Cl. G01N 33/48

(52) U.S. Cl. 436/116

(57) ABSTRACT

Pulmonary embolism in a human patient can be detected or confirmed by monitoring the level of endogenous NO in expired air, comparing the measured level with baseline values previously obtained for the same patient or baseline values representing a healthy population. The method can be used alone, or as a supplemental or adjunct step in traditional methods for diagnosing pulmonary embolism. Analysis of the obtained NO value, with consideration of the flow and resistance, can be used to indicate the severity of the pulmonary embolism.

### CROSS-REFERENCE TO RELATED APPLICATIONS

This application claims the benefit of Swedish Application No. 0402220-8, filed Sep. 14, 2004 under 35 USC Section 119.

### FIELD OF THE INVENTION

The present invention relates to steps performed in the diagnosis of pulmonary embolism, and in particular to a rapid, sensitive and non-invasive test assisting in the early detection and in the evaluation of the severity of pulmonary embolism, or functioning as one step in such diagnosis. The invention in particular relates to pulmonary thromboembolism, and the early detection thereof. The invention further encompasses methods for the pre- and post-operative surveillance or monitoring of mammals, preferably human patients, as well as methods for observing the pulmonary condition of such patients during operations.

### BACKGROUND OF THE INVENTION

Embolism

An embolus is a foreign object, a quantity of air or gas, a fat globule, a bit of tissue or tumour, or a piece of a thrombus that circulates in the bloodstream until it becomes lodged in a vessel, partially or completely obstructing blood flow.

Pulmonary embolism is a common disorder accompanied by a significant morbidity and mortality. Thromboembolism may either be acute through activation of the blood clotting system and disseminated intravascular coagulation, or occur at a later stage through the formation of thrombi in the pulmonary vessels or formation in the venous circulation with subsequent embolisation to the lung. The cause behind thrombi in the lung can also be so called thrombotisation, i.e. the formation of microthrombi in the circulation, triggered by tissue factors in the blood vessels. These microthrombi travel in the circulation until becoming trapped in the capillaries in the lung. It is estimated up to 40% of all cases of pulmonary embolism may be of this origin.

It is also estimated that pulmonary embolism is the main or at least a contributory cause of in-patient death. Swedish autopsy records indicate that pulmonary embolism is involved in about 20% of in-patient deaths. Pregnant women, and in particular women undergoing caesarean section; cancer patients; trauma victims, and patients undergoing surgery, e.g. orthopaedic surgery, are at risk. Further risk groups include, but are not limited to, individuals confined to bed rest or other types of confinement or restriction in the movement of the body of limbs, both during medical treatment or recovery from such treatment, or during transportation, e.g. air travel. Still further risk groups include, but are not limited to patients with infections, suffering from diseases or undergoing pharmaceutical treatments, disturbing the blood clotting system or the system for resolution of blood clots.

One special form of pulmonary embolism, pulmonary gas embolism, is a well-known consequence of surgery, trauma, diving and aviation, including the exploration of space. Another form, pulmonary thromboembolism, is caused when a thrombus or fat globule travels in the blood to the lungs as a result of trauma, surgery or dislodging of a thrombus or part thereof from another location in the body, e.g. in deep venous thrombosis (DVT).

Both pulmonary gas embolism and thromboembolism are known to cause severe haemodynamic and gas exchange abnormalities, including acute respiratory distress, pulmonary infarct and pleuritis.

The clinical diagnosis of pulmonary embolism is difficult and unreliable. Diagnosis methods currently used include chest X-ray, angiography, lung scintigraphy, perfusion/ventilation screening, and computed tomography (CT). In particular so-called spiral CT method is held to be reliable and sensitive. In this method, the patient is subjected to continuous X-ray exposure while being transported through a rotating fan beam. This provides improved three-dimensional contrast and spatial resolution compared to conventional computed tomography, where data is obtained and computed from individual sequential exposures. Angiography however remains the “gold standard” although the spiral CT method is increasingly used.

Among bioassays, the main one is the D-dimer antibody-based assay, where an increased level of D-dimer, a protein that is released into the circulation during the process of fibrin blood clot breakdown, is used as an indication of thrombotic disease. In practice, the final diagnosis will be based on many factors, including the patient's clinical signs, physical examination, and the results provided by one or more of the above methods.

The standard treatment of lung embolisms involves for example the administration of nasal oxygen, infusion of anticoagulantia and/or thrombolytic agents, and surgical intervention. Inhaled nitric oxide (NO) has been tried experimentally, but consensus has not been reached if such treatment is efficacious or not.

For more information on pulmonary embolism, see “Guidelines on diagnosis and management of acute pulmonary embolism”, Task Force on Pulmonary Embolism, European Society of Cardiology, European Heart Journal 2000;21:1301-1336.

Nitric Oxide

Nitric oxide (NO) is a molecule of importance in several biological systems, and is continuously produced in the lung and can be measured in ppb (parts per billion) in expired gas. The discovery of endogenous NO in exhaled air, and its use as a diagnostic marker of inflammation dates back to the early 1990-ies (See e.g. WO 93/05709; WO 95/02181). Today, the significance of exhaled, endogenous NO is widely recognised, and since a few years back, a clinical analyser is available on the market (NIOX®, the first tailor-made NO analyser for routine clinical use with asthma patients, AEROCRINE AB, Solna, Sweden).

In the summer of 1997 the European Respiratory Journal published guidelines (ERS Task Force Report 10:1683-1693) for the standardisation of NO measurements in order to allow their rapid introduction into clinical practice. Also the American Thoracic Society (ATS) has published guidelines for clinical NO measurements (American Thoracic Society, Medical Section of the American Lung Association: Recommendations for standardized procedures for the online and offline measurement of exhaled lower respiratory nitric oxide and nasal nitric oxide in adults and children—1999, in Am J Respir Crit Care Med, 1999; 160:2104-2117).

In early experiments attempting to elucidate the role of NO in respiratory gas, massive helium or air emboli were used to totally arrest the circulation in the lungs of test animals. The results indicated that increased levels of NO could be detected in exhaled air (Gustafsson L E, Leone A M, Persson M G, Wiklund N P, Moncada S: Endogenous nitric oxide is present in the exhaled air of rabbits, guinea pigs and humans. Biochem Biophys Res Commun 1991;181:852-7).

In 1999, Deem et al. published results indicating that hemodilution during venous gas embolisation improves gas exchange, without altering V(A)/Q or pulmonary blood flow distributions (Anesthesiology., 1999 Dec;91(6):1861-72). A continuous infusion of nitrogen through the left internal jugular vein at a rate of approximately 0.006 ml/kg min was applied to achieve embolisation. In the results, an increase of NO is recorded in embolised, hemodiluted anaemic test animals, but not in undiluted controls. The authors state that the difference between baseline and T1 VNO was statistically significant only for anaemic animals. In practice, no increase in NO was recorded for animals with normal hematocrit. Deem et al. also discuss the limitations of the model, venous gas embolisation using a continuous infusion of small bubbles, and states that it may be dissimilar to cases of air embolisation in the clinical setting, and that extrapolation of the data to clinical management is difficult.

The effect of vasodilator therapy was investigated in a canine model of acute pulmonary hypertension (Priebe, Am. J. Physiol. 255 (Heart Circ. Physiol, 24):H1232-H1239, 1998). In this study, pulmonary embolisation was simulated by injecting a suspension of finely chopped muscle tissue in saline containing 2000 U of heparin. Small volumes (0.5-2 ml) of the muscle suspension was injected repeatedly through a femoral vein catheter until the mean pulmonary arterial pressure had increased approximately threefold.

One objective of the present invention is to make available a method for early, sensitive and reliable diagnosis of pulmonary embolism, and in particular pulmonary thromboembolism. In the light of the inconclusive evidence presented in the prior art, it remains a problem to identify alternative markers, with the aim to find a reliable marker for non-invasive, rapid and sensitive diagnosis of pulmonary embolism, in particular pulmonary thromboembolism.

### SUMMARY OF THE INVENTION

The present inventors have surprisingly found that the level of endogenous nitric oxide (NO) in a sample of expired air can be used as an indication of pulmonary embolism, in particular pulmonary thromboembolism. Further analysis of the level of exhaled NO, e.g. at different flow velocities, and/or against different pressures, and the level of NO in relation to other exhaled gases can give additional information, e.g. as to severity of the embolisation.

The present invention is thus directed to a method of diagnosis of a pulmonary embolism in a human. The method of the invention includes detecting a level of endogenous NO in at least one sample of expired air taken from a human, and diagnosing whether the human has a pulmonary embolism based on the level of endogenous NO.

In the method of the invention, the diagnosing may include comparing the level of endogenous NO to a previously measured level of endogenous NO from the human or to a normalized level of NO representing a healthy population wherein the human is diagnosed as having a pulmonary embolism where the level or endogenous NO is elevated.

The present invention is further directed to a method for monitoring a patient during and after surgery. The method includes detecting a level of endogenous NO in at least one sample of expired air taken from the humans, and diagnosing whether the human has a pulmonary embolism based on the level of endogenous NO.

The diagnosing may include comparing the level of endogenous NO to a previously measured level of endogenous NO from the human or to a normalized level of NO representing a healthy population wherein the human is diagnosed as having a pulmonary embolism where the level or endogenous NO is elevated.

The methods of the invention may be used for monitoring a patient in intensive care, following surgery or trauma, or a combination thereof. Alternatively, the method of the invention may be utilized for monitoring a patient susceptible to pulmonary embolism.

In one format of the invention, at least one sample of expired air is/are taken, and the measured endogenous NO value is calculated to represent air from the peripheral airways.

In one another format of the invention, the level of NO is measured in at least two samples of expired air, obtained during one or more exhalations, wherein the one or more exhalations exhibit at least two different flow velocities.

In another format of the invention, the flow velocity and resistance is recorded, and the NO measurement are analysed to determine from what part of the lung or lungs the increased level of NO originates and thereby estimating the severity of the pulmonary embolism.

In another format of the invention, the ratio of exhaled CO₂ and NO is determined. In this format, an increase of NO and a simultaneous decrease of CO₂ is taken as an indication of pulmonary embolism.

In another format of the invention the amount of total exhaled NO is determined by measuring the exhalation flow and NO concentration during a predetermined period of time.

In one format of the invention the pulmonary embolism is pulmonary thromboembolism. In another format of the invention, the pulmonary embolism is pulmonary thrombotisation.

The present invention is further directed to a method of detecting haemolysis. The method includes detecting a level of endogenous NO in at least one sample of expired air taken from the human and diagnosing whether a haemolytic condition is present, based on the level of endogenous NO.

The present invention is further directed to a method wherein the level of NO is measured in at least two samples of expired air, obtained during one or more exhalations, wherein the one or more exhalations exhibit at least two different flow velocities.

### BRIEF DESCRIPTION OF THE DRAWINGS

The invention will be described in closer detail in the following description, examples, and attached drawings, in which

FIG. 1 shows the changes in mixed exhaled nitric oxide (FE_{NO}) upon muscle emboli challenge (time 0) in artificially-ventilated pentobarbital anaesthetised rabbits. In group 1 (n=6, open circles and bar, 58 mg kg⁻¹) and in group 2 with inhibited NO-production (n=4, L-NAME 30 mg kg⁻¹, closed circles and bars, 7.5 mg kg⁻¹). The bars show the infusion times. * indicates p<0.05 compared to control levels (time=−5)

FIG. 2 shows changes in end-tidal CO₂ (ETCO₂) upon muscle emboli challenge (time 0) in artificially-ventilated pentobarbital anaesthetised rabbits. Group 1 (n=6, open circles and bar, 58 mg kg⁻¹) and in group 2 with inhibited NO-production (n=4, L-NAME 30 mg kg⁻¹, closed circles and bars, 7.5 mg kg⁻¹). The bars show the infusion times. “¾” and “ 2/4” indicates survival in group 2 (L-NAME group) at the indicated time points.

FIG. 3 shows changes in mean arterial blood pressure (MAP) upon muscle emboli challenge (ME, time 0) in artificially-ventilated pentobarbital anaesthetised rabbits. Group 1 (n=6, open circles and bar, 58 mg kg⁻¹) and in group 2 with inhibited NO-production (n=4, L-NAME 30 mg kg⁻¹, closed circles and bars, 7.5 mg kg⁻¹). The horizontal bars labelled ME show the infusion times. “¾” and “ 2/4” indicates survival in group 2 (L-NAME group) at the indicated time points.

FIG. 4 shows changes in heart rate (HR) upon muscle emboli challenge (time 0) in artificially-ventilated pentobarbital anaesthetised rabbits. Group 1 (n=6, open circles and bar, 58 mg kg⁻¹) and in group 2 with inhibited NO-production (n=4, L-NAME 30 mg kg⁻¹, closed circles and bars, 7.5 mg kg⁻¹). The horizontal bars labelled ME show the infusion times for the muscle embolism challenge. “¾” and “ 2/4” indicates survival in group 2 (L-NAME group) at the indicated time points.

FIGS. 5 and 6 show arterial oxygen tension and haemoglobin oxygen saturation in rabbits challenged with Muscle embolus (unpretreated, 58 mg kg−1; n=6) as indicated by horizontal bar labelled ME.

FIGS. 7 and 8 show arterial carbon dioxide tension and pH in rabbits challenged with muscle embolus (unpretreated, 58 mg kg−1; n=6) as indicated by horizontal bar labelled ME.

FIG. 9 shows percent survival in unpretreated control animals (artificially-ventilated pentobarbital anaesthetised rabbits) (open circle, n=6 and 2 for 58 and 120 mg kg⁻¹ respectively) receiving muscle embolus challenge (ME) at 58 to 120 mg kg⁻¹, and in animals receiving 7.5 to 30 mg kg⁻¹ muscle embolus challenge after pre-treatment with L-NAME (30 mg kg⁻¹; filled circle, n=4, 2 and 2 for 7.5, 15 and 30 mg kg−1 respectively).

### DESCRIPTION

The present inventors have surprisingly shown that thromboembolism, as simulated with the muscle homogenate used in the examples, was associated with a rapid, sustained and significant increase of NO in expired air.

Although previous studies have indicated that gas embolisation, using either a massive helium bolus (Gustafsson et al., Biochem Biophys Res Commun 1991;181:852-7) or nitrogen microembolisation to hemodiluted animals (Deem et al., Anesthesiology., 1999 Dec;91(6):1861-72) influence exhaled endogenous NO, the results have hitherto been inconclusive.

Thus there was no basis to believe that the gas embolisation model would be representative for thromboembolism in vivo. The physical, chemical and physiological characteristics of a gas bubble, obstructing a blood vessel, and a thrombus, consisting of endogenous matter, are considerably different. In gas embolisation, the gas bubble creates a specific interface between gas and vascular endothelial tissue. In fact, the exact mechanism behind increased NO and embolisation is still not elucidated. Possibly the gas bubble exerts stress on the endothelial tissue by mechanical action. Another possibility is that reduced or obstructed blood circulation decreases excretion of CO₂ and therefore the alveolar concentrations of CO₂ are lower, which in turn has an effect on the NO production.

Cremona et al. (QJM. 1994 Sep;87(9):547-51) studied mixed expired nitric oxide (NO) production of the lungs of patients with primary pulmonary hypertension, to determine the relationship between NO production and the diffusion capacity of the lung. Results indicate that exhaled NO levels are reduced, as a reflection of the reduced blood capillary volume in patients with pulmonary hypertension.

In another study, Cremona et al. (Eur Respir J., 1995 Nov; 8(11):1883-5) measured exhaled NO in patients with hepatopulmonary syndrome, seen in severe chronic liver dysfunction. This occurs as a result of precapillary pulmonary arterial dilatation and arteriovenous communications. The molar rate of production of exhaled NO was raised almost threefold in the patients with hepatopulmonary syndrome compared with normal volunteers and with normoxaemic cirrhotic patients.

In an early work by Borland, Cox & Higenbottam (Thorax. 1993 Nov;48(11):1160-2) it was concluded that the circulation of blood stimulates NO production in capillary vessels in the lungs.

The published studies on exhaled endogenous NO indicate that an increased pulmonary blood flow would lead to a corresponding increase of NO levels in exhaled air, and that decreased or disturbed circulation would result in decreased NO levels. Interestingly, the present inventors have previously found that moderate decreases in blood flow through the lungs do not influence exhaled nitric oxide.

It was therefore not obvious that an effect of pulmonary embolism or gas embolism would be seen on exhaled NO in non-hemodiluted animals. Furthermore, blood flow through the lungs has been argued to be an important stimulus for NO generation from the endothelial cells in the pulmonary capillaries and that this endothelial NO contributes to exhaled NO. Therefore the possibility of a decrease in exhaled NO during partial blockade during embolisation would lie in the perspective of tentative outcomes of such a challenge.

The present inventors surprisingly found that pulmonary embolism causes a significant increase in exhaled NO, both during embolisation by muscle embolus, homogenised blood clots and during gas embolisation. Furthermore, the inventors showed that this increase is sustained for at least 60 min following embolisation muscle embolus, and based on this, suggest the novel possibility that this increase in exhaled NO can be used as an indicator of the embolisation. Even further, the inventors surprisingly showed that endogenous NO has a counteracting effect on the vascular effects, since they are much stronger when endogenous NO formation has been blocked, even to the extent that survival is less in the groups where NO formation was blocked.

One embodiment of the invention is using endogenous NO, determined in one or more samples taken from, and not returned to, expired air, as a marker for pulmonary embolism. This use can serve as an adjunct step in a diagnostic method, providing an indication of pulmonary embolism, or as a supplement or confirmation of a diagnosis reached using one or more alternative methods.

Another embodiment resides in the use of the level of exhaled NO relative to the level of another gas in exhaled air, e.g. CO₂, O₂, N₂ etc, as a marker for pulmonary embolism. Presently preferred is the determination of NO and CO₂, where an increase of NO and a simultaneous decrease of CO₂ is taken as an indication of pulmonary embolism.

Further, the rate of change in the level of NO and another gas, e.g. CO2, may also be indicative of the condition, its degree of severity, localization, the time passed since the formation of the embolism, a worsening of the patient's condition, or a recovery following pharmaceutical or surgical intervention, etc.

Further still, the total volume of exhaled NO can be estimated by measuring the exhaled volume and NO concentration. This may be useful in order to compensate for hyperventilation, a frequent symptom in patients suffering from pulmonary embolism.

Another embodiment involves the measurement of exhaled NO at different flow rates, and extrapolation of the results to give an indication as to the severity of the embolism, the size of the affected area and possibly also where in the lung, or lungs, the embolism is located.

The present invention thus makes available methods in the diagnosis of pulmonary embolism in a human patient, wherein the level of NO in a sample or samples of expired air is measured, and where an elevated level as compared to previous measurements in the same patient, or to normalized values representing a healthy population, is taken as an indication of pulmonary embolism.

The invention also makes available methods in the monitoring of patient health during and after surgery, wherein the level of NO in a sample or samples of expired air is measured, and where an elevated level as compared to previous measurements in the same patient, or to normalized values representing a healthy population, is taken as an indication of pulmonary embolism.

The invention further makes available methods in the monitoring of patients in intensive care, following surgery or trauma, or a combination thereof, wherein the level of NO in a sample or samples of expired air is measured, and where an elevated level as compared to previous measurements in the same patient, or to normalized values representing a healthy population, is taken as an indication of pulmonary embolism.

The invention also makes available methods in the monitoring of a patient susceptible to pulmonary embolism, wherein the level of NO in a sample or samples of expired air is measured, and where an elevated level as compared to previous measurements in the same patient, or to normalized values representing a healthy population, is taken as an indication of pulmonary embolism.

In all of the above embodiments, it is preferred that the sample or samples of expired air is/are taken, or the measured NO value calculated, so as to represent air from the peripheral airways.

It may be of advantage to measure the level of NO in at least two samples of expired air, obtained during one or more exhalations, wherein said one or more exhalations exhibit at least two different flow velocities. Therefore, a preferred embodiment of the invention includes the measurement of the level of NO in at least two samples of expired air, obtained during one or more exhalations, wherein said one or more exhalations exhibit at least two different flow velocities. This is achieved e.g. using a variable flow resistance in the flow path.

In the above embodiments, the pulmonary embolism is preferably pulmonary thromboembolism.

Alternatively, in the above embodiments, the pulmonary embolism is preferably pulmonary thrombotisation.

One particular embodiment is the screening of patients seeking medical advice due to symptoms normally associated with pulmonary embolism, or symptoms giving the physician the reason to suspect pulmonary embolism. Another embodiment is the monitoring of patients remitted to medical care or hospitalised based on symptoms associated with pulmonary embolism, or symptoms giving the physician the reason to suspect pulmonary embolism. Another embodiment is the regular monitoring, either at a doctor's office, a hospital or at home, of patients with a previous history of thrombosis, or susceptible to thrombosis due to medication, disease, as a consequence of therapy, genetic predisposition or general state of health.

The invention naturally includes the above embodiments in both an in-patient and out-patient setting.

Yet another embodiment of the invention is using the inventive method as a security measure in connection to scheduled surgery, and in particular in surgery where pulmonary thrombosis is a frequent, potential or possible side-effect. Examples include but are not limited to orthopaedic surgery, caesarean section, angiografting, anti-thrombotic intervention, organ transplantations, liposuction, cosmetic surgery, etc. According to this embodiment, the baseline NO is preferably recorded by one or more NO measurements taken before the scheduled surgery, to establish a baseline value for the individual patient.

Alternatively, a baseline value, based on values normally associated with a healthy population, is used. It is also conceived that the rate of increase or the NO profile is monitored, wherein a rapid increase is indicative of acute pulmonary thromboembolism.

Similarly, the rate of the increase, or decrease, of exhaled endogenous NO can be used to obtain more data, which can be linked to the severity and the course of events in a case of pulmonary thromboembolism, such as the improvement or worsening of the patient's condition, the effects of therapeutic treatments etc.

Another embodiment is the monitoring of patients remitted to an intensive care unit, either following surgery, trauma or acute illness. In such cases, where previously recorded NO levels may not be available, a baseline value, based on values normally associated with a healthy population, is used.

Yet another embodiment of the invention is the use of endogenous NO in expired air as an indication of haemolysis, haemolytic disorders or crisis, such as encountered in surgery, trauma, drowning, and various diseases. Surgery in this case includes all intervention in the body, as well as situations where blood contacts non-endogenous surfaces, such as dialysis and other examples of blood filtration, artificial circulation such as a heart-lung machine, cardiac prosthesis, implants, etc. Diseases associated with haemolysis include antigen-antibody reactions, metabolic abnormalities etc. Particular examples include, but are not limited to, haemolytic anaemia, thalassemia, malaria, sickle cell anaemia, hereditary spherocytosis, spherocytic anaemia, etc.

In situations of haemolysis, the endogenous NO is rapidly absorbed by haemoglobin and a decreased level of NO will be detected in samples of exhaled air.

In the context of the present invention, the terms “increased” and “decreased” means a property of a measured value, when compared to values previously obtained for the same individual, or a value, representative for a healthy population.

Advantages

The early detection of pulmonary embolism, afforded by the method according to the present invention, makes it possible to introduce therapy, either in the form of anticoagulantia or other pharmaceutical agents, or in the form of assisted breathing or supplementary oxygen, or surgical intervention, at an early stage, preventing exacerbation of the condition.

Surprisingly, NO—in the concentrations involved—has been shown to be a highly specific marker for pulmonary embolism, unlike other gaseous components, previously suggested for this purpose.

The methods according to the present invention are well suitable both in a bedside (point-of-care) setting, for use in a doctor's office, and for home use.

The methods according to the present invention are useful alone, or as a complement or supplement to existing methods, e.g. to confirm a diagnosis obtained using physical examination or other methods.

One particular advantage of the methods according to the invention is that they are easily applicable to intensive care settings, as well as to the operating room.

An important advantage is that all embodiments of the invention represent methods, which are non-invasive, and involve no exposure to radiation, contrast agents, transport or other manipulation of the patient. The inventive methods are therefore particularly suitable for pregnant women, children, weakened patients, patients undergoing operation or intensive care etc.

The inventive method can be put in practice using existing, commercially available apparatuses, e.g. the NIOX® NO-analyser (AEROCRINE AB, Solna, Sweden).

The invention will now be described in closer detail in the following non-limiting examples.

### EXAMPLES

The inventive method was confirmed by the present inventors in vivo, in an animal model, using muscle homogenate to simulate thromboembolism. Experiments testing a blood clot homogenate and thrombin infusion were also performed.

Anaesthesia and Initial Surgical Procedures

The experiments were approved by the local animal ethics committee. Male white New Zealand rabbits (n=20, body weight 2.456±0.086 kg) were anaesthetised via an ear vein with sodium pentobarbital, 6 mg ml⁻¹ in normal saline, 40-60 mg kg⁻¹. The animals were placed in supine position and tracheotomised just below the cricoid cartilage to allow mechanical ventilation using a tracheal cannula with an outer diameter of 5 mm. The animals were ventilated by a Harvard Apparatus rodent ventilator (model 683, Harvard Apparatus, South Natick, Mass., USA). The ventilator was supplied with NO-free air using a charcoal filter (110×11 cm). Ventilation rate was 40 min⁻¹ at constant volume where the tidal volume was initially adjusted to keep the end-tidal CO₂ at 4.5-5.3% as determined by a ventilatory monitor (Oscar-Oxy, Datex, Helsinki, Finland) sampling gas (150 ml min⁻¹, 15-20% of minute ventilation) from one of two side-arms connected to the tracheal cannula, and using a de-humidifying tube. The minute ventilation was 0.64-0.96 l min⁻¹. To the other side arm a pressure transducer (Statham, Hato Rey, Puerto Rico) was connected thus monitoring the insufflation pressure. The gas from the ventilator outlet was led through a switching valve to either of two beakers creating a positive end-expiratory pressure (PEEP) of 1-2 cmH₂O or 4-5 cmH₂O. During the experiment the gas flow was altered between the lower PEEP (9 min) and the higher PEEP (1 min) with an interval of totally 10 min. A continuous infusion containing glucose (24.3 g l⁻¹), dextran 70 (26.5 g l⁻¹), NaHCO₃ (6,2 g l⁻¹), sodium pentobarbital (4.1 g l⁻¹) and pancuronium bromide (98 mg ml⁻¹) was administrated at a rate of 5 ml kg⁻¹ h⁻¹ via the same ear vein by means of a Terumo STC-521 syringe pump (Terumo Corp., Tokyo, Japan). A heparinised catheter was inserted in the left common carotid artery for blood pressure and heart rate recordings (pressure transducer, Staham, Hato Rey, Puerto Rico), and arterial blood sampling. Another catheter was inserted in the right jugular vein for drug and muscle emboli administration. Body temperature was maintained at 37-38.5° C. by means of a heating pad connected to a thermostat. The muscles from the anterior compartment of the right lower hind limb were resected and placed in normal saline. Hereafter the animals were allowed a 30-60 min intervention-free period to obtain stable circulatory conditions and stable concentrations of expired NO.

NO Measurements in Exhaled Air

NO concentration, in mixed exhaled gas, was continuously measured by means of a chemiluminescence based system (NIOX®, AEROCRINE AB, Solna, Sweden) sampling at 100 ml min⁻¹ at the end of a mixing chamber connected to the ventilator exhaust. The full mixing of expired air thus measured on was intermittently checked by monitor CO₂ concentration in the same chamber. In a few experiments, gas for NO measurement was sampled from the trachea at the same point as for tidal CO₂ measurements, thus yielding breath by breath NO concentrations. Calibration was done using certified NO standard gas in nitrogen (AGA Specialgas, Lidingö, Sweden).

Preparation of Muscle Emboli

The resected muscle tissue was cleared from all visible connective tissue and then homogenized and dissolved in normal saline to a concentration of 0,1 g muscle ml⁻¹. 50 IE heparin was added to the mixture. The homogenate was filtered through a filter (500 μm) to prevent clotting in the three way stop-cock.

Experimental Protocol

The animals were divided into two groups; 1) one group receiving a high dose (58-120 mg kg⁻¹) muscle homogenate and 2) a second group receiving the nitric oxide inhibitor L-NAME (30 mg kg⁻¹) 40 min before challenge with lower doses (30 to 7.5 mg kg⁻¹) muscle homogenate, since initial pilot experiments indicated a marked enhancement of emboli effects after L-NAME pre-treatment. Blood samples were collected and analysed for blood gases and acid-base status (ABL 300, Radiometer A/S, Copenhagen, Denmark) before L-NAME administration (group 2, time=−50 min) and shortly before muscle emboli challenge (group 1 and 2, time=−5 min). The muscle homogenate was infused by means of an infusion pump (CMA/100, Microinjection Pump, Carnegie Medicine AB, Stockholm, Sweden) with a flow of 150 μl kg⁻¹ min⁻¹ via a three way stop-cock into a carrier flow (864 Syringe Pump, Univentor LTD., Zejtun, Malta) of 150 μl kg⁻¹ min⁻¹ normal saline through the jugular vein catheter until full muscle emboli dose for each group was received. Arterial blood samples were collected and analysed at 10 min, 20 min, 40 min and 60 min after embolisation. NO concentration in exhaled gas, end-tidal CO₂, heart rate, mean arterial pressure and insufflation pressure was continuously monitored on a Grass Polygraph (Grass Instruments Co, Quincy, Mass., USA) during the experiments. Blood gases and acid-base status were analysed by a Radiometer ABL 300 blood gas analyser (Radiometer A/S, Copenhagen, Denmark).

Drugs

Heparin was purchased from Kabi Vitrum, Stockholm, Sweden, pancuronium bromide (Pavulon®) was from Organon, Oss, Holland, sodium pentobarbital was from Apoteksbolaget, Stockholm, Sweden and dextran 70 (Macrodex®) was from Pharmalink, Spåanga, Sweden. L-NAME (N^{G}-nitro-L-arginine methyl ester) and routine chemicals were purchased from Sigma Chemical Company, St Louis, Mo., USA.

Statistics

Statistical data are given as mean and standard error of the mean (SEM). Statistical significance was calculated by means of repeated measurements ANOVA on ranks with Dunnet's post hoc analysis. P<0,05 was assigned as significance difference. All statistical calculations were done by using a computer program (SigmaStat, Jandel, San Rafael, Calif., USA).

Results

Status of Animals Before Muscle Emboli Challenge

Group 1 (control): After the intervention-free period but before muscle emboli challenge FE_{NO}, ETCO₂, MAP and HR stabilised at 22.3±1.9 ppb, 5.2±0.1 kPa, 109.4±4.5 cmH₂O and 283±12 beats min⁻¹ respectively (n=6, time=−5). The relevant blood gas parameters were all normal (Table 1).

TABLE 1
Summary of blood gas and acid-base status in rabbits receiving Muscle embolus
challenge without pre-treatment (Group 1, 58 mg kg-1) or before and during
pre-treatment with L-NAME (Group 2, 7.5 mg kg-1). N = 4-6

| | Time (min) | | | | | |
|---|---|---|---|---|---|---|
| | −40 | −5 | 10 | 20 | 40 | 60 |
| Group 1: | | | | | | |
| Parameter | | | | | | |
| PaO₂ (kPa) | | 9.5 ± 0.4 | 6.1 ± 0.6 | 5.7 ± 0.3 | 5.9 ± 0.3 | 6.4 ± 0.3 |
| PaCO₂(kPa) | | 4.6 ± 0.1 | 5.5 ± 0.3 | 6.1 ± 0.3 | 6.0 ± 0.2 | 5.7 ± 0.2 |
| pH | | 7.48 ± 0.02 | 7.39 ± 0.04 | 7.29 ± 0.05 | 7.24 ± 0.05 | 7.24 ± 0.06 |
| Group 2: | | | | | | |
| (L-NAME) | | | | | | |
| PaO₂ (kPa) | 9.1 ± 0.8 | 8.6 ± 0.6 | | | | |
| PaCO₂(kPa) | 4.6 ± 0.1 | 4.3 ± 0.2 | | | | |
| pH | 7.48 ± 0.01 | 7.50 ± 0.02 | | | | |

Group 2 (L-NAME): FE_{NO}, ETCO₂, MAP and HR stabilised at 20.9±1.0 ppb, 4.9±0.1 kPa, 105±6 cmH₂O and 286±11 beats min⁻¹ respectively after intervention free-period but before L-NAME infusion (n=4, time=−40). After L-NAME infusion but before muscle emboli challenge (time=−5), FE_{NO}, ETCO₂, MAP and HR changed to 0 ppb, 4.8±0.2 kPa, 121±8.2 cmH₂O and 314±34 beats min⁻¹, although these changes did not reach statistical significance. The relevant blood gas parameters were all normal at these time points.

Effects of Muscle Emboli Challenge

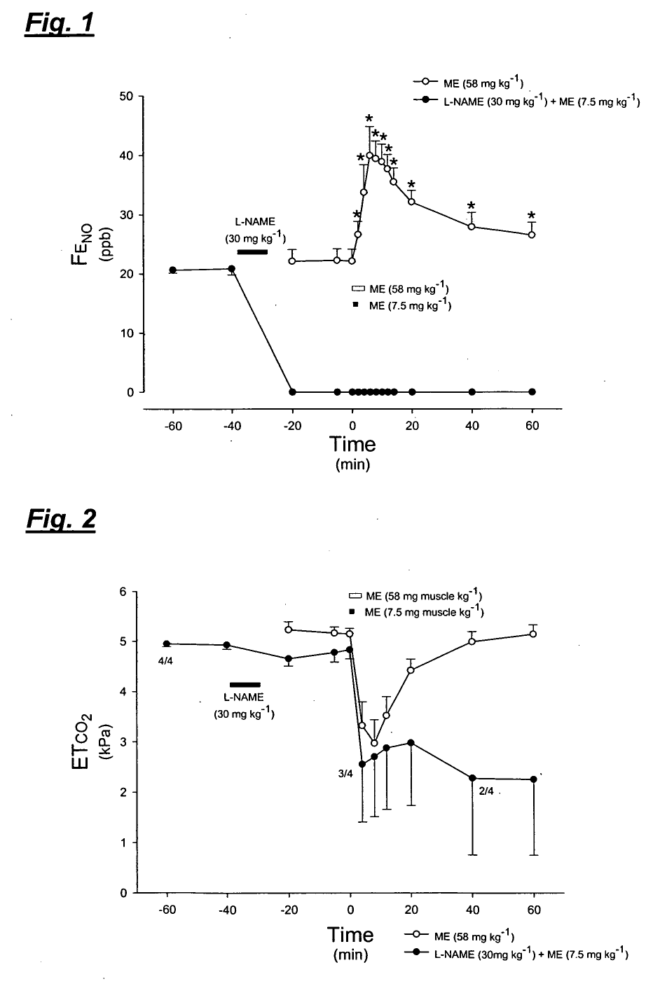

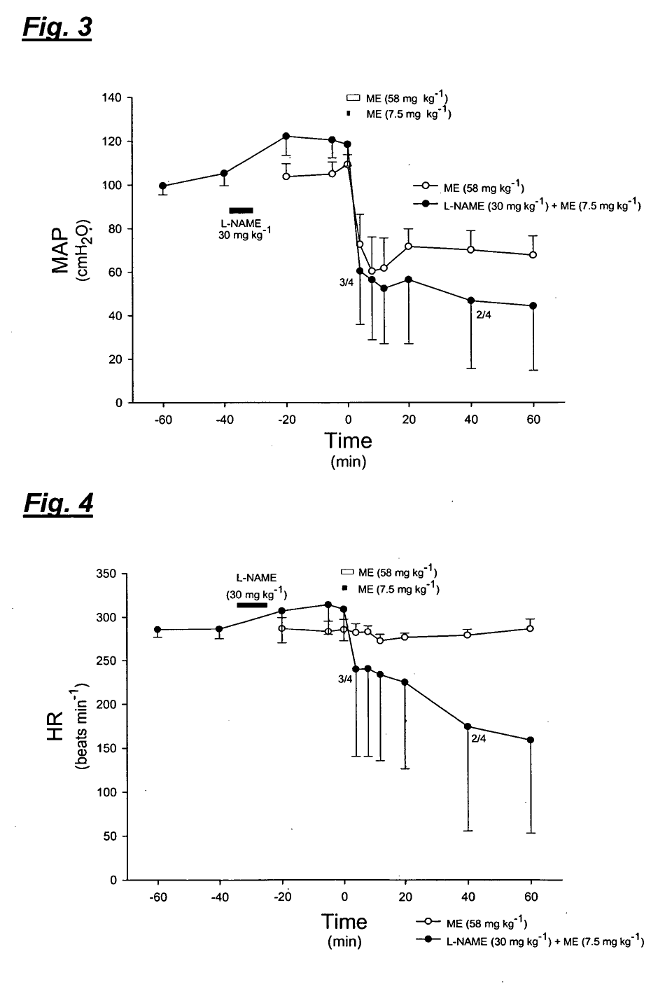

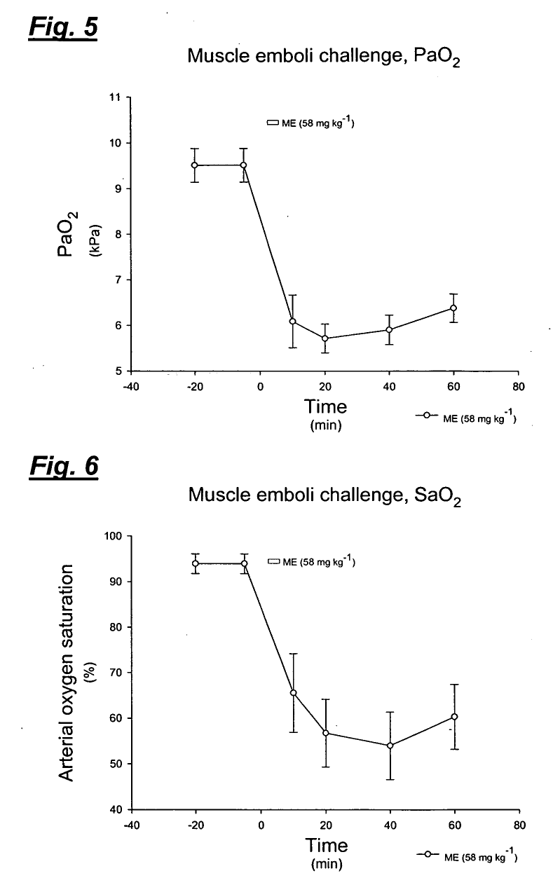

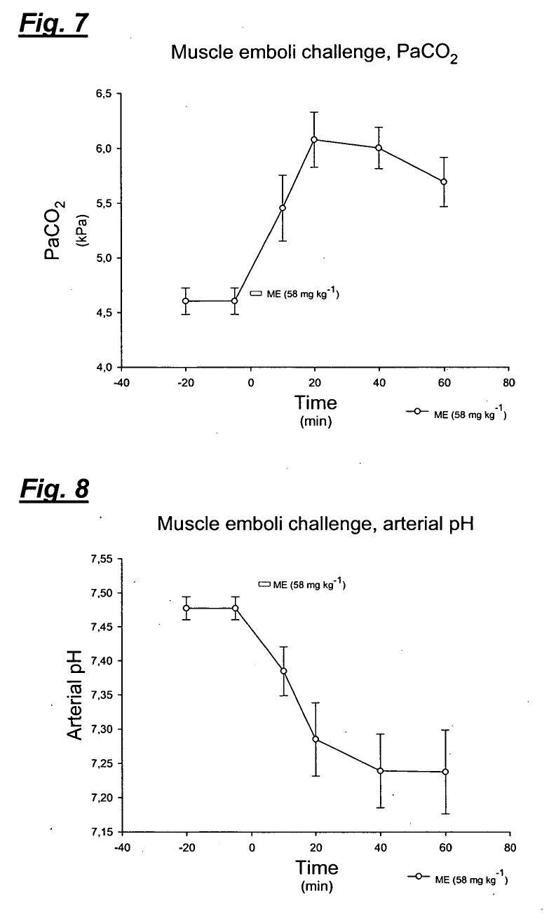

Group 1) Upon muscle emboli infusion (58 mg kg⁻¹), FE_{NO} rapidly increased from basal control levels of 22.3±1.9 ppb in exhaled gas. The FE_{NO} value peaked at 40.0±4.9 ppb within 6 min, and then slowly decreased but was still significantly elevated (26.5±2.2 ppb) after 60 min compared to baseline conditions (time=−5, FIG. 1). Parallel to the NO increase there were significant decreases in ETCO₂ (lowest observed value was at 8 min, 3.0±0.5 kPa, FIG. 2) and MAP (lowest observed value was at 8 min, 60.4±15.8 cmH₂O, FIG. 3). HR did not change significantly (FIG. 4). PaO₂ (FIG. 5) was significantly decreased and PaCO₂ (FIG. 7) was significantly increased until 40 min, and pH (FIG. 8) was significantly decreased after 60 min. Blood gas values are shown in table 1.

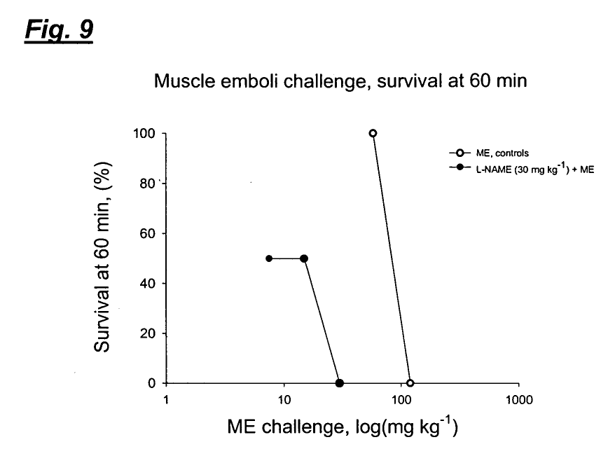

Group 2) Pilot experiments with L-NAME pre-treatment indicated that lower doses of muscle embolus challenge than in group 1 had to be used to obtain any survival beyond 10 min after challenge, see also FIG. 9. Upon muscle emboli infusion with 7.5 to 30 mg kg−1 homogenate one animal died within 5 min and another animal died within 30 min, whereas two animals survived 60 min. All four rabbits are included in the statistics until 60 min and therefore produce large SEM values. ETCO₂ decreased significantly (lowest observed value was at 4 min, 2.6±1.1 kPa; see FIG. 2). Mean arterial pressure (MAP; FIG. 3) decreased significantly, and the response was biphasic with one drop at 12 min (52.4±25.5 cmH₂O) and the lowest observed value at 60 min (44.4±29.6 cmH₂O). Though not significant, HR decreased (FIG. 4).

During the course of experiments, a trial was performed in the above described animal model, and following the same procedures, but using blood drawn for the test animals, coagulated and homogenised, in order to more closely simulate real life pulmonary thromboembolism. The blood clot homogenate was re-infused in the animals in the same fashion as the muscle homogenate. This homogenate however exhibited marked haemolysis when given intravenously, and surprisingly a 50% decrease in exhaled NO was recorded. This indicates that exhaled endogenous NO is an indicator of haemolysis.

Another experiment was performed with infusion of thrombin (10-250 Units) as pro-thrombotic challenge. Also in this experiment, an increase in exhaled NO was obtained when measurement of cardiovascular parameters indicated an activation of a thrombotic event.

Clinical Experiments

The inventors have scheduled clinical experiments to be performed in an emergency ward setting. Patients hospitalised with symptoms normally associated with pulmonary embolism (chest pains, insufficient oxygenation, hyperventilation etc) are to be subjected to an analysis of the gaseous components in exhaled air, with particular emphasis on the determination of the concentration of endogenous NO in exhaled air. When pulmonary embolism is diagnosed by existing methods, the pattern of exhaled NO will be recorded over time, and in response to treatment in these patients. A clinical NO analyser (NIOX™, Aerocrine AB, Solna, Sweden) will be made available by the manufacturer.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1. A method of diagnosis of a pulmonary embolism in a human comprising:
detecting a level of endogenous NO in at least one sample of expired air taken from said human, and
diagnosing whether said human has a pulmonary embolism based on said level of endogenous NO.

2. The method of claim 1, wherein said diagnosing comprises comparing said level of endogenous NO to a previously measured level of endogenous NO from said human or to a normalized level of NO representing a healthy population wherein said human is diagnosed as having a pulmonary embolism where said level or endogenous NO is elevated.

3. A method for monitoring a patient during and after surgery, comprising
detecting a level of endogenous NO in at least one sample of expired air taken from said human, and
diagnosing whether said human has a pulmonary embolism based on said level of endogenous NO.

4. The method of claim 1, wherein said diagnosing comprises comparing said level of endogenous NO to a previously measured level of endogenous NO from said human or to a normalized level of NO representing a healthy population wherein said human is diagnosed as having a pulmonary embolism where said level or endogenous NO is elevated.

5. A method for monitoring a patient in intensive care, following surgery or trauma, or a combination thereof, comprising a method according to claim 1.

6. A method for monitoring a patient susceptible to pulmonary embolism comprising a method according to claim 1.

7. A method according to claim 1, wherein said at least one sample of expired air is/are taken, and the measured endogenous NO value calculated to represent air from the peripheral airways.

8. The method according to claim 7, wherein the level of NO is measured in at least two samples of expired air, obtained during one or more exhalations, wherein said one or more exhalations exhibit at least two different flow velocities.

9. The method according to claim 7, wherein the flow velocity and resistance is recorded, and the NO measurement analysed to determine from what part of the lung or lungs the increased level of NO originates and thereby estimating the severity of the pulmonary embolism.

10. The method according to claim 1, wherein the ratio of exhaled CO₂ and NO is determined and wherein an increase of NO and a simultaneous decrease of CO₂ is taken as an indication of pulmonary embolism.

11. The method according to claim 1, wherein the amount of total exhaled NO is determined by measuring the exhalation flow and NO concentration during a predetermined period of time.

12. The method according to claim 1, wherein the pulmonary embolism is pulmonary thromboembolism.

13. The method according to claim 1, wherein the pulmonary embolism is pulmonary thrombotisation.

14. A method in the detection of haemolysis, comprising
detecting a level of endogenous NO in at least one sample of expired air taken from said human, and
diagnosing whether a haemolytic condition is present, based on said level of endogenous NO.

15. The method of claim 14, wherein a decreased level as compared to previous measurements in the same patient, or to normalized values representing a healthy population, is taken as an indication of haemolysis.

16. The method according to claim 14, wherein the sample or samples of expired air is/are taken so as to represent, or the measured NO value calculated, to represent air from the peripheral airways.

17. The method according to claim 14, wherein the level of NO is measured in at least two samples of expired air, obtained during one or more exhalations, wherein said one or more exhalations exhibit at least two different flow velocities.
